(19) United States
(12) Patent Application Publication
(10) Pub. No.: US 2010/0022552 A1
(43) Pub. Date: Jan. 28, 2010

(54) KMUP-1 CAPABLE OF TREATING HYPERTENSION

(75) Inventor: Ing-Jun CHEN, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: Kaohsiung Medical University, Kaohsiung City (TW)

(21) Appl. No.: 12/572,519

(22) Filed: Oct. 2, 2009

Related U.S. Application Data
(63) Continuation-in-part of application No. 11/857,483, filed on Sep. 19, 2007.

(30) Foreign Application Priority Data
Jun. 15, 2007 (TW) 096121950

(51) Int. Cl. A61K 31/496; A61P 9/12; A61P 9/00

(52) U.S. Cl. 514/252.16

(57) ABSTRACT

A method for treating a spontaneous hypertension or a cardiomyocyte hypertrophy is provided. The method comprises a step of administering to a mammal a therapeutically effective amount of a compound of 7-[2-[4-(2-chlorobenzene)piperazinyl]ethyl]-1,3-dimethyl xanthine.

### CROSS-REFERENCE TO RELATED APPLICATIONS

This application is a Continuation-In-Part of co-pending application Ser. No. 11/857,483 filed on Sep. 19, 2007, and for which priority is claimed under 35 U.S.C.§120; and this application claims priority of Application No. 96121950 filed in Taiwan on Jun. 15, 2007 under 35 U.S.C.§119; the entire contents of all are hereby incorporated by reference.

### FIELD OF THE INVENTION

The present invention relates to a theophylline-based compound capable of increasing eNOS and inhibiting iNOS expressions, and more particularly to a compound of 7-[2-[4-(2-chlorobenzene)piperazinyl]ethyl]-1,3-dimethyl xanthine capable of treating a spontaneous hypertension.

### BACKGROUND OF THE INVENTION

U.S. Pat. No. 6,979,687 disclosed a serial of theothylline-based compounds, KMUP-1 and KUMP-2, having a minimum inhibition on phosphodiester (PDE), and capable of activating the soluble guanynyl cyclase (sGC). The inhibition on PDE enhances the concentration of cGMP and the activation on sGC promotes the production of cGMP. cGMP molecule modulates the regulation of the NO-releasing relevant proteins relaxes the blood vessels. Therefore, it has been proven in the mentioned patent that KMUP-1 contributes to the relaxation of the blood vessels of the corpus cavernosal in the penis.

KMUP-1 has been reported to increase eNOS and inhibit iNOS expression. Several studies have found that eNOS and iNOS are involved in the ischemic protection and the preservation of vascular contractility. iNOS was found to be reduced in cardiac hypertrophy induced by aortic banding, and antioxidant treatment was found to restore the loss of iNOS and abolish the cardiac hypertrophy. Mice undergoing transverse aortic constriction and iNOS-deficient mice have been reported to be less prone to cardiac hypertrophy. Clearly, eNOS can regulate impaired endothelial NO bioactivity in left ventricular hypertrophy (LVH), and the eNOS inhibitor N-omega-nitro-1-arginine (L-NNA) can reduce vascular relaxation through the NO-cGMP pathway, worsening LVH and decreasing survival.

eNOS and iNOS are important for NO/cGMP production, and phosphodiesterase (PDE)-5A is the major enzyme for the cGMP hydrolysis. The balance between the expressions of these three enzymes decides the amount of cGMP produced in cardiomyocytes. The PDE-5 inhibitor sildenafil has been shown to have cardioprotective and anti-hypertrophic activities by blocking the degradation of cGMP. It has been suggested that cGMP-enhancing sildenafil might be used to treat cardiac hypertrophy and increase myocardial dilator reserve. In ischemia/reperfusion injury, sildenafil reduced the infarct size and facilitated post-ischemic ventricle recovery. Sildenafil also suppressed cardiomyocyte hypertrophy in hearts exposed to pressure-overloading induced by aortic constriction. Enhanced cGMP prevents the hypertrophic signaling and antagonizes cyclic adenosine monophosphate (cAMP) by increasing protein kinase G (PKG). Because mitogen-activated protein kinases (MAPK), also known as ERK1/2, were found by many studies to be a critical mediator of cardiac hypertrophy, we hypothesized that ERK1/2 would be activated in the LVH of spontaneous hypertensive rats (SHRs). A treatment strategy which increases eNOS/cGMP/PKG and reduces iNOS expression in cardiovascular system, such as with KMUP-1 or sildenafil, might be used to reverse ERK1/2 expression in hypertensive cardiac hypertrophy.

From the above description, it is known whether KMUP-1 is effective in the spontaneous hypertension has become a major problem waited to be solved. In order to overcome the drawbacks in the prior art, another pharmaceutical activity of KMUP-1 is provided. The particular design in the present invention not only solves the problems described above, but also is easy to be implemented. Thus, the invention has the utility for the industry.

### SUMMARY OF THE INVENTION

In the present invention, we first show that KMUP-1 is superior to sildenafil for the treatment of the hypertensive LVH via the measurements of the changes in blood pressure, heart weight, survival and protein expression in SHRs and WKY rats treated with either KMUP-1 or sildenafil.

In accordance with one aspect of the present invention, a method for treating a spontaneous hypertension is provided. The method comprises a step of administering to a mammal a therapeutically effective amount of a compound of 7-[2-[4-(2-chlorobenzene)piperazinyl]ethyl]-1,3-dimethyl xanthine.

Preferably, the administration is performed by at least one of an oral injection and an intraperitoneal injection.

Preferably, the administration is performed by the oral injection at a dosage of 10-30 mg/kg/day for 28 days.

Preferably, the administration is performed by the intraperitoneal injection at a dosage of 10-30 mg/kg/day for 28 days.

Preferably, the administration is performed by the intraperitoneal injection at a dosage of 0.5 mg/kg/day for 10 days.

Preferably, the method further comprises a step of administering the compound with a pharmaceutically effective carrier thereof.

In accordance with another aspect of the present invention, a method for treating a cardiomyocyte hypertrophy is provided. The method comprises a step of administering to a mammal a therapeutically effective amount of a compound of 7-[2-[4-(2-chlorobenzene)piperazinyl]ethyl]-1,3-dimethyl xanthine.

Preferably, the method further comprises a step of administering the compound with a pharmaceutically effective carrier thereof.

Preferably, the cardiomyocyte hypertrophy is a hypertensive left ventricular hypertrophy.

Preferably, the administration is performed by at least one of an oral injection and an intraperitoneal injection.

Preferably, the administration is performed by the oral injection at a dosage of 10-30 mg/kg/day for 28 days.

Preferably, the administration is performed by the intraperitoneal injection at a dosage of 10-30 mg/kg/day for 28 days.

Preferably, the administration is performed by the intraperitoneal injection at a dosage of 0.5 mg/kg/day for 10 days.

The above aspects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a schematic diagram showing the effects of KMUP-1 on tail mean artery blood pressure (MAP) changes in SHRs and WKY rats. *, p<0.05, **, p<0.01 versus the SHRs-CTL group.

FIG. 2 is a schematic diagram showing the effects of KMUP-1, sildenafil, or either in combination with L-NNA on the cumulative survival within 10 days.

FIGS. 3A and 3B are schematic diagrams showing the effects of KMUP-1, sildenafil, or either in combination with L-NNA on the heart weight (HW)/body weight (BW) ratio in SHRs. A: *, p<0.05 versus the WKY-CTL group and ⁺, p<0.05 versus the SHR-CTL group; B: ^{#}, p<0.05 and ^{##}, p<0.01 versus the SHR-CTL group, and **, p<0.01 versus the SHR-L-NNA group.

FIGS. 4A and 4B are schematic diagrams showing the effects of KMUP-1, sildenafil or either in combination with L-NNA on eNOS expression in the left ventricle. A: *, p<0.05 and **, p<0.01 versus the WKY-CTL group, and ⁺, p<0.05 versus the SHR-CTL group; B: ^{##}, p<0.01 versus the SHR-CTL group, **, p<0.01 versus the SHR-L-NNA group.

FIGS. 5A and 5B are schematic diagrams showing the effects of KMUP-1, sildenafil or either in combination with L-NNA on iNOS expression in the left ventricle. A: **, p<0.01 versus the WKY-CTL group and ⁺, p<0.05 versus the SHR-CTL group; B: ^{##}, p<0.01 versus the SHR-CTL group and *, p<0.05 versus the SHR-L-NNA group.

FIGS. 6A and 6B are schematic diagrams showing the effects of KMUP-1, sildenafil or either in combination with L-NNA on nitric oxide concentration (NOx) expression in the left ventricle. A: **, p<0.01 versus the WKY-CTL group and ⁺, p<0.05 versus the SHRs-CTL group; B: ^{#}, p<0.05 versus the SHR-CTL group, and *, p<0.05 versus the SHR-L-NNA group.

FIGS. 7A and 7B are schematic diagrams showing the effects of KMUP-1, sildenafil or either in combination with L-NNA on PKG expression in the left ventricle. A: *, p<0.05 and **, p<0.01 versus the WKY-CTL group, and ⁺, p<0.05 versus the SHR-CTL group; B: ^{##}, p<0.01 versus the SHR-CTL group and **, p<0.01 versus the SHRs-L-NNA group.

FIGS. 8A and 8B are schematic diagrams showing the effects of KMUP-1, sildenafil or either in combination with L-NNA on the calcineurin A (Cn A) protein expression in the left ventricle. A: **, p<0.01 versus the WKY-CTL group and ⁺⁺, p<0.01 versus the SHR-CTL group; B: ^{##}, p<0.01 and ^{#}, p<0.05 versus the SHR-CTL group, and **, p<0.01 versus the SHRs-L-NNA group.

FIGS. 9A and 9B are schematic diagrams showing the effects of KMUP-1, sildenafil or either in combination with L-NNA on the ERK1/2 protein expression in the left ventricle. A: **, p<0.01 versus the WKY-CTL group and ⁺⁺, p<0.01 versus the SHR-CTL group; B: ^{##}, p<0.01 and ^{#}, p<0.05 versus the SHR-CTL group and **, p<0.01 versus the SHRs-L-NNA group.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention provides a chemical compound, KMUP-1, having a pharmaceutical activity of anti-hypertension. The detailed description for the pharmaceutical experimental results of KMUP-1 is provided as below.

Pharmaceutical Trials

1. The Preparation for the Present Chemical Compound

The preparation of KMUP-1 has been disclosed in U.S. Pat. No. 6,969,687, and thus it will not be mentioned again in the present invention.

2. Long-Term and Short-Term Treatments

Two experimental phases, part 1 for long-term and part 2 for short-term treatment, were carried out in this invention. Hereinafter “p.o.” is referred to the oral administration and “i.p.” is referred to the intraperitoneal administration.

In part 1, long-term treatment, SHRs and WKY (Non-genomic disease type, WKY) rats (n=10 in each group) received KMUP-1 (10 or 30 mg/kg/day) for 28 days. Nine-week-old WKY rats were divided into WKY-control and WKY-KMUP-1 groups. The WKY-control rats received vehicle and the WKY-KMUP-1 group received KMUP-1 (vehicle containing, 10 mg/kg/day). SHRs were divided into SHRs-control and SHR-KMUP-1 groups. The SHRs-control group received vehicle only and the SHRs-KMUP-1 group received KMUP-1 (vehicle containing, 10 or 30 mg/kg/day).

In part 2, for short-term treatment, nine-week-old SHRs were randomly assigned into 6 groups (n=15 in each group). The SHR-control group received intraperitoneal saline vehicle injection daily for 10 days. The SHRs-KMUP-1 group received intraperitoneal KMUP-1 in a vehicle-containing dose of 0.5 mg/kg/day. The sildenafil group received intraperitoneal sildenafil in a vehicle-containing dose of 0.7 mg/kg/day. The SHRs-L-NNA group received L-NNA in drinking water (20 mg/L). The SHRs-L-NNA+KMUP-1 group received both L-NNA (20 mg/L) in drinking water and KMUP-1 (vehicle-containing 0.5 mg/kg/day). The SHRs-L-NNA+sildenafil group received both L-NNA in drinking water and sildenafil (vehicle-containing 0.7 mg/kg/day).

3. Testing of Blood Pressure

Nine-week-old male SHRs and WKY rats, with an elevated basal blood pressure of 150 mmHg and a non-elevated blood pressure of 120 mmHg on average were chosen for the experiments. Rats were measured for systolic blood pressure and heart rate without anesthetic using the indirect tail cuff method with a rat tail manometer-tachometer (MK-2000 Storage Pressure Meter, Muromachi Kikai Co., LTD, Japan). The rats were restrained in a plexiglass holder at a temperature of 37° C. for 15-20 min to raise their body temperatures in the 28-day experiment. Blood pressure measurements were followed in the treated and untreated groups over the 28-day course of the experiment. The increase in temperature leads to dilation of the caudal artery, which allowed us to easily detect the pressure pulse. In all cases at least three consecutive measurements were obtained and the average was reported as the systolic blood pressure. Blood pressure changes in the 10-day experiment were not shown.

4. Survival and Heart Weight Indices

The number of survivors in each group was recorded daily until the end of study. The cumulative survival rate was determined by the equation: 10−total number of dead rats/10, from day 1 to day 10. The heart was perfused with saline, and the heart weight and body weight were recorded. The heart weight index was calculated by dividing the heart weight by the body weight.

5. Plasma Nitrite Test

A thoracic artery was cannulated, hepanized for collection of blood in heparin-coated sample tubes, and tubes were centrifuged at 2500 g for 15 min at 4° C. The plasma samples were incubated with nitrate reductase to reduce nitrates to nitrites, and the final concentration of NOx was determined by adding Griess reagent to the sample and measuring the absorbance at 540 nm. NOx concentration was expressed as μM and calculated using a standard curve for nitrite.

6. Western Blot Analysis

Expression of proteins, obtained from the aortas and the ventricles treated with KMUP-1 or sildenafil and from the control groups, were measured by Western blot. Mouse or rabbit monoclonal antibodies to eNOS (Upstate, N.Y., U.S.A.), sGCα (Sigma-Adrich, Calif., U.S.A.), PKG (Calbiochem, San Diego, Calif., U.S.A, and the loading control protein β-actin (Sigma-Adrich, Mo.) were used in the Western blot analyses.

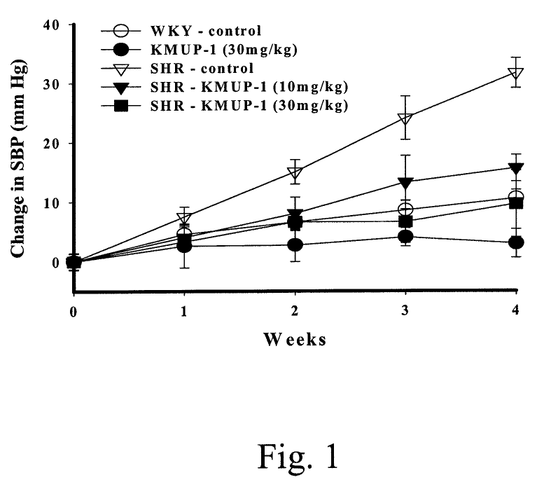

Please refer to FIG. 1, which shows the effects of KMUP-1 on tail mean artery blood pressure (MAP) changes in SHRs and WKY rats. The respective amounts, 10 mg/kg and 30 mg/kg, of KMUP-1 are orally administered to SHR(S) and WKY (W) rats with eight weeks old. As shown in FIG. 1, MAP changes in SHRs were inhibited more than WKY rats by KMUP-1 (10 and 30 mg/kg, p.o.) compared to vehicle control. In WKY and SHRs rats, the control basal levels of systolic MAP were 151±3.9 and 124.4±6.2 mmHg and increased to 181.6±5.7 and 134.6±3.2 mmHg, respectively, from week 9 to 12 (data not shown). In WKY rats, treatment with KMUP-1 (10 mg/kg/day, p.o.) for 28 days did not significantly reduce the blood pressure or changes in blood pressure from week 9 to 12. In SHRs, treatment with KMUP-1 (10 and 30 mg/kg/day, p.o.) for 28 days dose-dependently decreased the development of blood pressure. KMUP-1 (0.5 mg/kg/day, 28 days, i.p.) did not affect MAP changes in SHRs (data not shown).

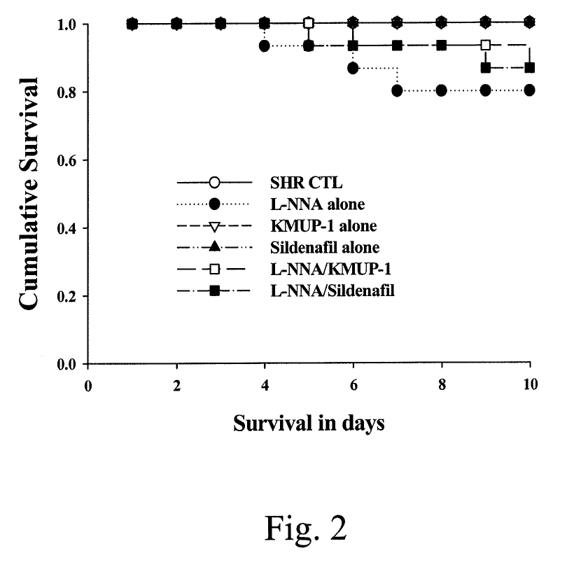

Please refer to FIG. 2, which shows the effects of KMUP-1, sildenafil, or either in combination with L-NNA on the cumulative survival within 10 days. Kaplan-Meier survival plots of SHRs after the 10-day experiment are shown. At the termination of the study, the survival rates of control SHRs and KMUP-1- and sildenafil-treated groups were all 100%. The SHRs-L-NNA+KMUP-1 group and the SHRs-L-NNA+sildenafil group had the same 87% survival rate (13/15). The survival rate of the SHRs-L-NNA group was 80% (12/15).

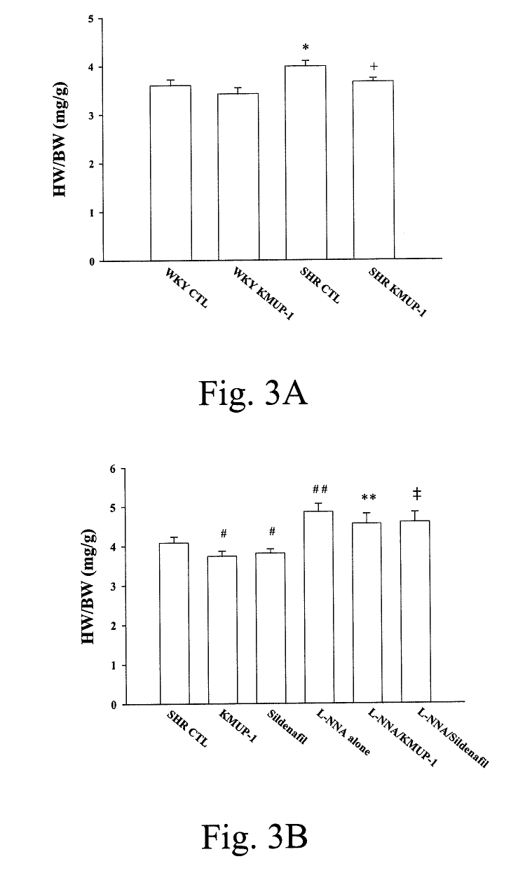

Please refer to FIGS. 3A and 3B, which show the effects of KMUP-1, sildenafil, or either in combination with L-NNA on the heart weight (HW)/body weight (BW) ratio in SHRs. Each bar represents the mean±S.E.M. As shown in FIG. 3A, SHRs had a significantly greater increase in heart weight index than the WKY rats (p<0.05). As shown in FIG. 3B, the heart weight/body weight ratio was significantly higher in the SHRs-L-NNA group than in the untreated SHRs; particularly, intra-peritoneal injection of KMUP-1 (0.5 mg/kg/day) and sildenafil (0.7 mg/kg/day) for 10 days reduced the heart weight indices (mg/g) in SHRs and in L-NNA-treated SHRs.

In the 28-day experiment, KMUP-1 (10 mg/kg/day, i.p.) significantly increased the expression of eNOS, sGC and PKG, respectively, in the aorta of WKY rats. KMUP-1 (10 or 30 mg/kg/day, p.o.) also dose-dependently increased expression of eNOS, sGC and PKG in SHRs (data not shown). In contrast, expression of iNOS was sharply increased in SHR-CTL rats, but not in the WKY-CTL group. Treatment with KMUP-1 (30, mg/kg/day, i.p.) significantly decreased the expression of iNOS (data not shown).

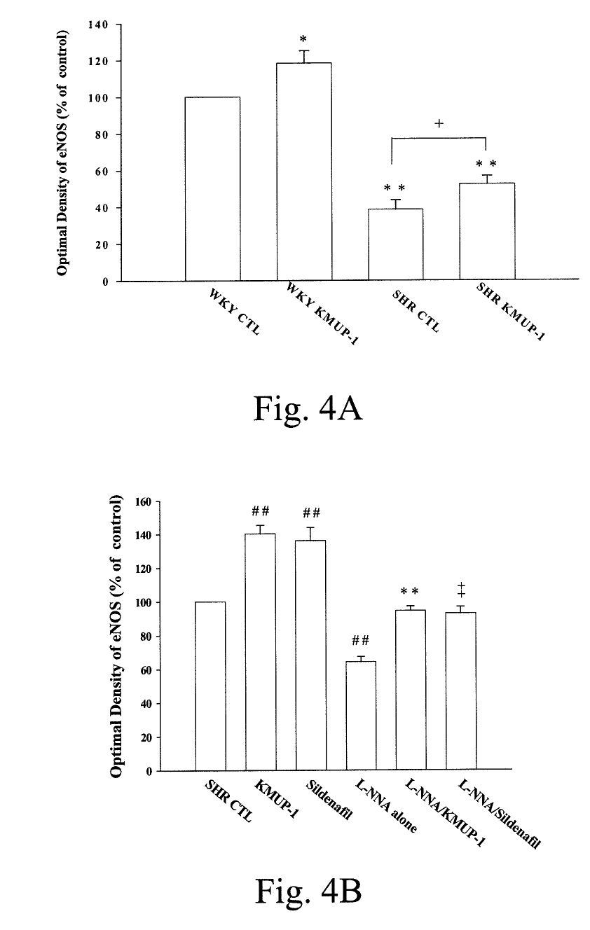

Please refer to FIGS. 4A and 4B, which show the effects of KMUP-1, sildenafil or either in combination with L-NNA on eNOS expression in the left ventricle. Densitometry analyses are presented as the relative ratio of eNOS protein/β-actin protein. Each value represents the means±S.E.M. In the 28-day experiment, expression of eNOS in SHRs and WKY rats was compared with the vehicle control (CTL). Oral administration of KMUP-1 (10 mg/kg/day) significantly increased ventricular eNOS expression in the SHR and WKY groups (FIG. 4A; p<0.05). eNOS expression in SHR-CTL rats was significantly different from that in the WKY-CTL group (p<0.01). In the 10-day experiment, both KMUP-1 (0.5 mg/kg/day, i.p.) and sildenafil (0.7 mg/kg/day, i.p.) increased the expression of eNOS in the SHR-KMUP-1 and SHR-sildenafil groups and in the SHR-L-NNA group (FIG. 4B; p<0.01). In conclusion, the treatment with KMUP-1 or sildenafil significantly increased the expression of eNOS (FIG. 4B; p<0.01).

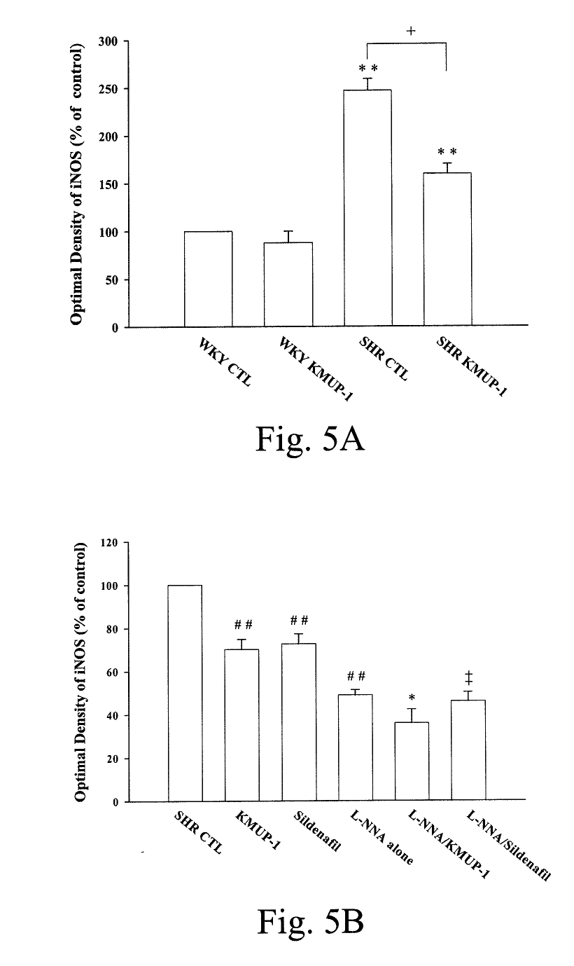

Please refer to FIGS. 5A and 5B, which show the effects of KMUP-1, sildenafil or either in combination with L-NNA on iNOS expression in the left ventricle. Densitometry analyses are presented as the relative ratio of iNOS protein/β-actin protein. Each value represents the means±S.E.M. In the 28-day experiment, the expression of iNOS in SHR-CTL rats was greater than that in the WKY-CTL group (FIG. 5A; p<0.01). Increased expression of iNOS was significantly lowered by the treatment with KMUP-1 (10 mg/kg/day) in SHRs (p<0.01), but not in the WKY-KMUP-1 group. As shown in FIG. 5B, in the 10-day experiment, treatment with KMUP-1 (0.5 mg/kg/day, i.p.) or sildenafil (0.7 mg/kg/day, i.p.) significantly decreased the expression of iNOS in SHRs (p<0.01). L-NNA sharply decreased the expression of iNOS compared to the SHR-CTL group (p<0.01). This was not altered by co-treatment with KMUP-1 or sildenafil, compared to the SHR-L-NNA group, although these two groups were significantly different from the SHR-CTL group (p<0.01).

This study demonstrates that eNOS and iNOS are involved in sildenafil's and KMUP-1's anti-hypertrophic effects in SHRs. In these animals, the attenuation of cardiac hypertrophy by long-term intra-peritoneal sildenafil and KMUP-1 was accompanied by the increased expression of eNOS and the decreased expression of iNOS. We suggest that an early increase of eNOS might prevent later worsening of LVH by reducing the expression of iNOS and production of more peroxynitrate in pressure-overloaded cardiac endothelial cells. In WKY rats, we suggest that KMUP-1 significantly increases eNOS and insignificantly affects iNOS expression.

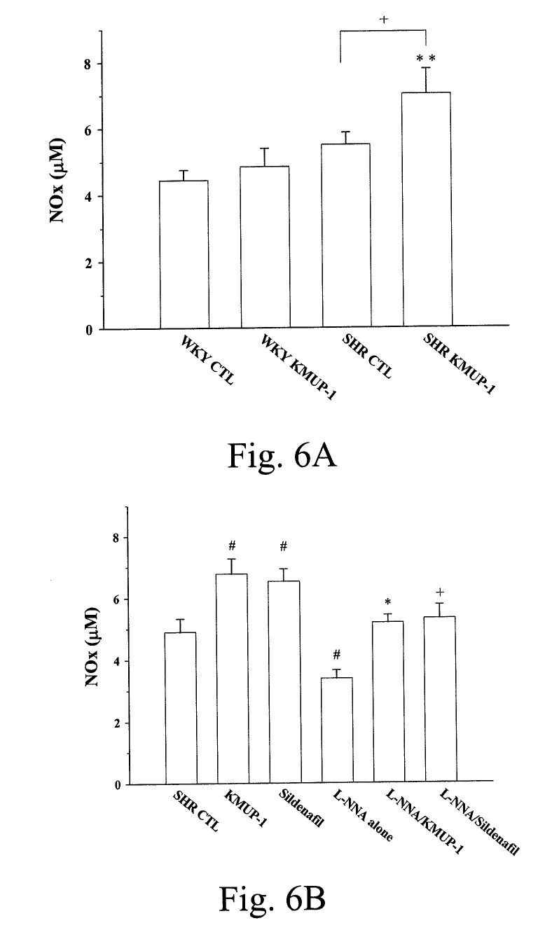

Please refer to FIGS. 6A and 6B, which show the effects of KMUP-1, sildenafil or either in combination with L-NNA on nitric oxide concentration (NOx) expression in the left ventricle. NOx production was measured by the level of NO metabolites, nitrite (NO₂) and nitrate (NO₃⁻) in rat plasma. Each value represents the means±S.E.M. The plasma of rats that received oral KMUP-1 (10 mg/kg/day) for 28 days is sampled to measure NOx. As seen in FIG. 6A, there were no differences among the plasma NOx levels in the WKY, WKY-KMUP-1 and SHRs-control (SHR CTL) groups, whereas SHRs treated with KMUP-1 were found to have significantly higher plasma NOx levels than the SHR controls (p<0.05). As shown in FIG. 6B, in the 10-day experiment for SHRs, treatment with KMUP-1 (SHR-KMUP-1 group) and sildenafil (SHR-sildenafil group) significantly increased the plasma NOx concentration of the SHR-CTL group (p<0.05), while treatment with L-NNA significantly reversed the release of NOx (p<0.05). Moreover, treatment with KMUP-1 or sildenafil also increased NOx in L-NNA treated SHRs (p<0.05).

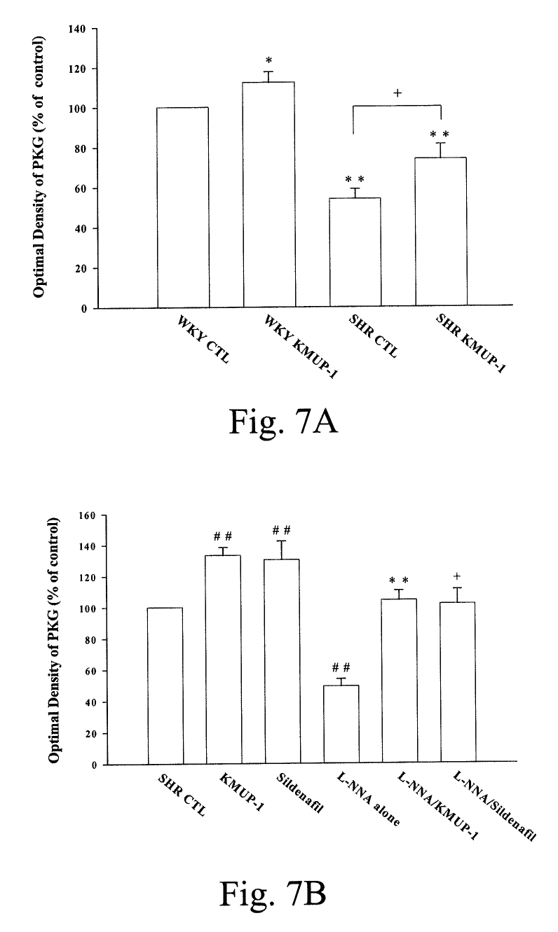

Please refer to FIGS. 7A and 7B, which show the effects of KMUP-1, sildenafil or either in combination with L-NNA on PKG protein expression in the left ventricle. Densitometry analyses are presented as the relative ratio of PKG protein/β-actin protein. In FIG. 7A, the expression of PKG in the left ventricle after 28 days of treatment is measured. It is found that KMUP-1 (10 mg/kg/day, p.o.) significantly increased PKG in WKY rats and also in SHRs, compared to the WKY-CTL and SHR-CTL group, respectively (FIG. 7A, p<0.05). The SHR-CTL group showed sharply lower PKG expression than the WKY-CTL group (FIG. 7A). In the 10-day experiment, as shown in FIG. 7B, treatment with KMUP-1 (0.5 mg/kg/day, i.p.) or sildenafil (0.7 mg/kg/day, i.p.) significantly increased the expression of PKG in the SHR-KMUP-1 and SHR-sildenafil groups (p<0.01), but treatment with L-NNA alone (SHR-L-NNA group) significantly inhibited the expression of PKG, compared to the SHR-CTL group (p<0.01). In contrast, treating the SHR-L-NNA group with KMUP-1 or sildenafil significantly increased the expression of PKG (p<0.01). This study showed that KMUP-1 and sildenafil increased the expression of PKG in the aorta and ventricles of SHRs. Treatment with L-NNA sharply decreased expression of PKG in SHR-L-NNA. However, co-treatment with L-NNA increased this expression. We suggest that accumulation of PKG by sildenafil or KMUP-1 in SHRs can avoid the increase in heart weight index.

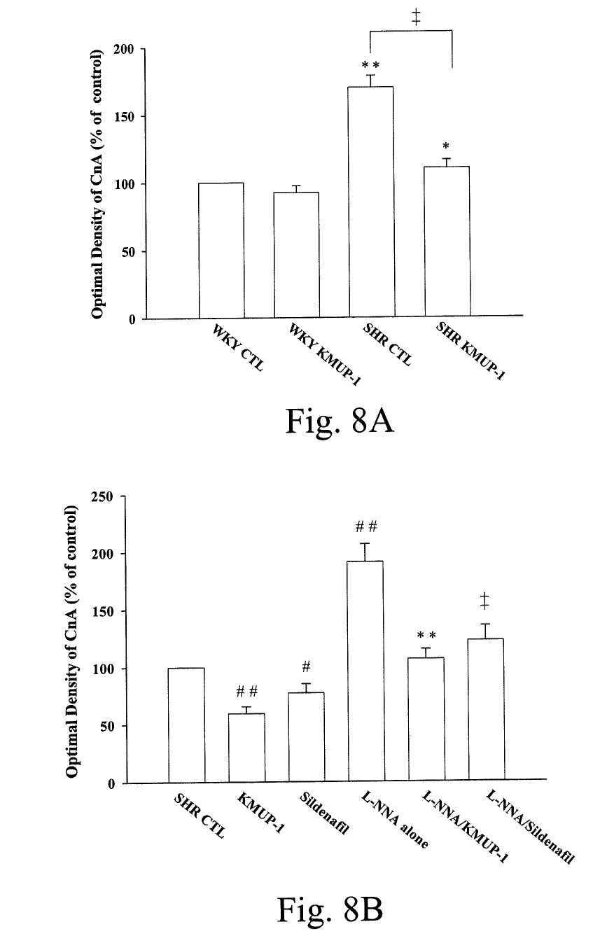

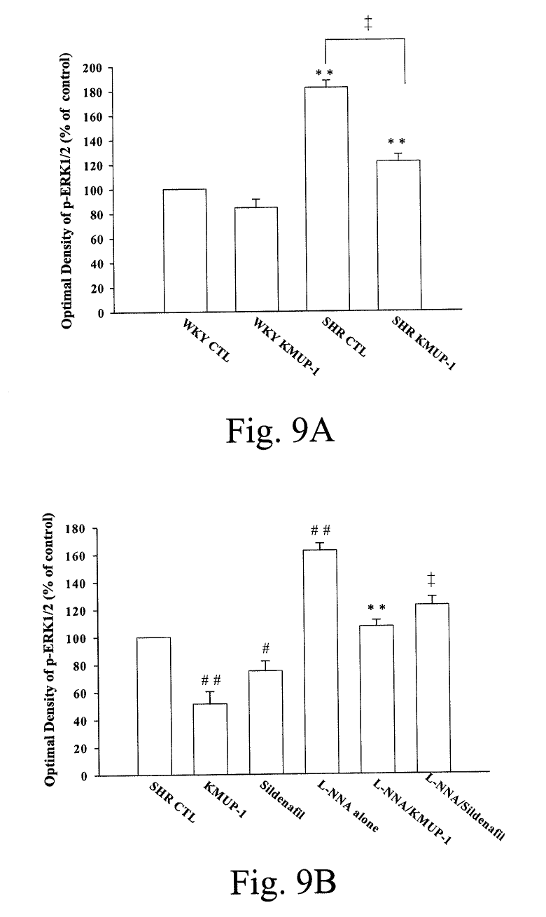

FIGS. 8A and 8B show the effects of KMUP-1, sildenafil or either in combination with L-NNA on the calcineurin A (Cn A) protein expression in the left ventricle. FIGS. 9A and 9B show the effects of KMUP-1, sildenafil or either in combination with L-NNA on the p-ERK1/2 protein expression in the left ventricle. As seen in FIGS. 8A and 9A, SHRs had higher expressions of Cn A and phosphorylated ERK1/2 after 28 days than control WKY rats (p<0.01). Treatment with KMUP-1 (10 mg/kg/day, p.o.) significantly attenuated expression of Cn A and phosphorylated ERK1/2 in SHRs (p<0.01), but not in WKY rats.

In FIGS. 8B and 9B, SHR rats were used in the experiments. As shown, compared to the SHR CTL group, both KMUP-1 and sildenafil significantly inhibited expression of Cn A and p-ERK1/2 in SHRs (p<0.01 and p<0.05 respectively). KMUP-1 was more potent than sildenafil in inhibiting p-ERK1/2 expression (p<0.01). SHRs treated with L-NNA for 10 days were found to have increased expression of Cn A and phosphorylated ERK1/2 (p<0.01), which could be significantly prevented by KMUP-1 (0.5 mg/kg/day) and sildenafil (0.7 mg/kg/day) (p<0.01). ERK1/2, which is activated by Cn A and growth factors, plays an important role in cell proliferation and differentiation. Treatment with KMUP-1 and sildenafil attenuated both Cn A expression and ERK1/2 phosphorylation in SHRs, indicating they both have cardioprotection properties even under hypertension stress.

As mentioned in the above, it could be known that LVH in SHRs is characterized by an increased heart weight/body weight ratio and ventricular expression of ERK1/2, Cn A and iNOS, and the present invention indicates that KMUP-1 enhances aortic and ventricular eNOS/PKG/NOx and prevents ventricular iNOS, Cn A and ERK1/2 expression to provide satisfactory ventricular anti-hypertrophy benefits, in addition to its anti-hypertension effects. Therefore, the expression of eNOS/iNOS/CnA/ERK1/2 in this study can serve as an early sub-clinical signature of KMUP-1's ventricular anti-hypertrophy effects in the treatment of hypertension.

KMUP-1 is more potent than sildenafil in inhibiting ventricular ERK1/2 expression, suggesting a different signaling mechanism in the cardiovascular system and KMUP-1 is more effective than sildenafil for treating hypertensive LVH. KMUP-1 enhances cardiovascular eNOS/sGC/PKG expression, leading to the inhibitions of MAP and LVH in SHRs. These results indicate the usefulness of KMUP-1 as a possible alternate to sildenafil for the treatment of hypertension and LVH. KMUP-1 anti-hypertrophic signaling is initiated via an eNOS-potentiation of the cGMP/PKG pathway and subsequent Cn A/ERK1/2-suppression under hypertension conditions.

Accordingly, the present invention can effectively solve the problems and drawbacks in the prior art, and thus it fits the demand of the industry and is industrially valuable.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for treating a spontaneous hypertension, comprising a step of administering to a mammal a therapeutically effective amount of a compound of 7-[2-[4-(2-chlorobenzene)piperazinyl]ethyl]-1,3-dimethyl xanthine.

2. A method as claimed in claim 1, wherein the administration is performed by at least one of an oral injection and an intraperitoneal injection.

3. A method as claimed in claim 2, wherein the administration is performed by the oral injection at a dosage of 10-30 mg/kg/day for 28 days.

4. A method as claimed in claim 2, wherein the administration is performed by the intraperitoneal injection at a dosage of 10-30 mg/kg/day for 28 days.

5. A method as claimed in claim 2, wherein the administration is performed by the intraperitoneal injection at a dosage of 0.5 mg/kg/day for 10 days.

6. A method as claimed in claim 1, further comprising a step of administering the compound with a pharmaceutically effective carrier thereof.

7. A method for treating a cardiomyocyte hypertrophy, comprising a step of administering to a mammal a therapeutically effective amount of a compound of 7-[2-[4-(2-chlorobenzene)piperazinyl]ethyl]-1,3-dimethyl xanthine.

8. A method as claimed in claim 7, further comprising a step of administering the compound with a pharmaceutically effective carrier thereof.

9. A method as claimed in claim 7, wherein the cardiomyocyte hypertrophy comprises at least one of a hypertensive left ventricular hypertrophy and a right ventricular hypertrophy.

10. A method as claimed in claim 7, wherein the administration is performed by at least one of an oral injection and an intraperitoneal injection.

11. A method as claimed in claim 10, wherein the administration is performed by the oral injection at a dosage of 10-30 mg/kg/day for 28 days.

12. A method as claimed in claim 10, wherein the administration is performed by the intraperitoneal injection at a dosage of 10-30 mg/kg/day for 28 days.

13. A method as claimed in claim 10, wherein the administration is performed by the intraperitoneal injection at a dosage of 0.5 mg/kg/day for 10 days.
